(19) United States
(12) Patent Application Publication
(10) Pub. No.: US 2004/0225217 A1
(43) Pub. Date: Nov. 11, 2004

(54) Fingertip ultrasound medical instrument

(76) Inventors: James W. Voegele, Cincinnati, OH (US); Robert P. Gill, Mason, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/777,740

(22) Filed: Feb. 12, 2004

Related U.S. Application Data
(60) Provisional application No. 60/447,543, filed on Feb. 14, 2003.

(51) Int. Cl. A61B 8/00; A61N 7/00

(52) U.S. Cl. 600/439

(57) ABSTRACT

Disclosed is a minimally invasive surgical instrument that may be used in hand-assisted laparoscopic surgeries. The device is an ultrasonic transmitter and receiver and may be mounted directed on a surgeon's fingertip and inserted through an incision to allow the surgeon to monitor the operational field during a surgical procedure. The device may be used in combination with tactile feedback or other means of alerting the surgeon of the presence of blood vessels or arteries, for example, to provide the surgeon with an improved tactile sense of the surgical field.

### CROSS REFERENCE TO RELATED APPLICATIONS

The present application claims the benefit of U.S. Provisional patent application serial No. 60/447,543, filed on Feb. 14, 2003, the contents of which are hereby incorporated herein by reference.

The present application is also related to U.S. patent applications, attorney docket no. END-5016NP, Ser. No. [______] and END-5017NP, Ser. No. [______] filed concurrently herewith.

### FIELD OF THE INVENTION

The present invention relates in general to the performance of a variety of surgical steps or procedures during surgical operations and, more particularly, to methods and apparatus for utilizing ultrasonic sensing as an integral part of such surgical procedures to expedite and facilitate their performance and to extend a surgeon's sense of “feel” within a body cavity.

### BACKGROUND OF THE INVENTION

Two operations that commonly can be performed to advantage using hand assisted laparoscopic surgery (“HALS”) techniques are nephrectomy and bowel surgical repair. In both instances, a hand port is used in conjunction with one or more cannulas (trocars) that permits introduction of a combination illuminating and viewing instrument and a number of different endoscopic surgical instruments. The endoscopic instruments perform surgical steps or procedures required to complete the surgical operation prior to removing the cannulas and closing the relatively small openings required for their insertion.

A problem in using certain surgical instruments that is particularly apparent during endoscopic surgery is the lack of the surgeon's sense of feel and easy access to all internal body cavity locations. In non-endoscopic surgery (i.e. open surgery), a surgeon can normally easily verify the identification of structures or vessels within a conventional open surgery incision. In particular in the two noted operations, the surgeon normally uses the sense of feel to verify the nature of visually identified operational fields.

In a gall bladder operation, for example, the bile duct must be distinguished from a blood vessel that passes close to the duct. Also, the locations of blood vessels must be determined in the repair of an abdominal hernia using endoscopic surgery since such repair is performed by stapling a section of polymeric mesh material to the inside of the abdominal wall. The material securing staples must be placed to ensure that a blood vessel is not stapled during the repair.

The identification of blood vessels during endoscopic surgical operations has been addressed in the prior art. For example, in U.S. Pat. No. 4,770,185 issued to Silverstein et al, an ultrasonic probe is disclosed wherein pulsed ultrasonic energy is used in a catheter to identify both venous and arterial blood flows. A resulting Doppler signal is used to drive a loudspeaker such that the sense of hearing is used in place of the surgeon's sense of feel.

With the advance represented by HALS procedures there is a need for improved ultrasonic monitoring that can take advantage of the increased freedom created by having a hand inside the body cavity.

The present invention overcomes the disadvantages of the prior art and provides the surgeon with a cost effective, yet efficiently flexible medical instrument.

### SUMMARY OF THE INVENTION

This need is met by the methods and apparatus of the present invention wherein an ultrasonic sensing system is incorporated into a surgical device attached to a surgeon's hand, and more specifically to a surgeon's fingertip such that the surgical instrument is used to monitor an operational field.

In one aspect the surgical instrument is useful in minimally invasive surgery where the access to the surgical site is provided by a hand port. The surgical instrument may be manipulated within the surgeon's hand or the instrument may be slidably attached to the surgeon's finger and work as an extension of the surgeon's fingertip.

In one aspect of the invention, the distal end of the finger device angularly supports the ultrasonic transmitter to aim the ultrasonic transmitter at the operational field and similarly angularly supports the ultrasonic receiver at the operational field.

In one embodiment, the finger device comprises an ultrasonic transmitter/receiver for assessing the operational field off the pad of the first digit of a finger although an embodiment may be made to accommodate any digit. In the illustrated embodiments, the ultrasonic means operates at a frequency of approximately 20 megahertz. To extend the shelf life and conserve power during usage of battery-operated combinations in accordance with the present invention, power control means are coupled to the circuit means by a pressure switch mounted in conjunction with the ultrasonic means for connecting power to the circuit means only while the finger device is pressed against an operational field to activate. The wires connected to the sensor follow the surgeons arm, exiting the body port and connect to the associated circuitry.

To enable the ultrasonic sensing systems to be mobile, circuitry for performing ultrasonic sensing is preferably enclosed in housings worn by the surgeon, although off-surgeon configurations are optional. Wiring, run along the surgeon's arm connects the circuitry to transducers formed in or mounted on the distal ends of the hand device. The transducers direct ultrasonic energy to the operational fields defined by the distal ends of the finger device and receive ultrasonic energy reflected from the operational fields. Acoustic lenses, angularly oriented transducer mounts or a combination of the two may direct the transmission and receipt of ultrasonic energy within the operational field.

In another aspect of the invention signals representative of the tissue or contents of the operational field of a surgical instrument and generated by the ultrasonic sensing system are used to alerting the surgeon. The alert means may take a variety of forms, such as an audible signal generator or a tactile transducer for tactilely signaling the surgeon. The tactile transducer is mounted for access by the surgeon within the finger device. In this way, the present invention extends a surgeon's sense of feel for performance of surgical procedures, particularly HALS procedures. The sensitivity of the ultrasonic sensing system can be adjusted to prevent activation of the alerting means for background signal levels. The level of the alerting signal, whether audible or tactile, can also be adjusted. In one aspect of the invention the alert is a tactile transducer means coupled to the internal surface of the surgical instrument for tactilely communicating to the surgeon's fingertip thereby extending the surgeon's sense of feel. Alternately, the alert may comprise an audible signal generator such as a speaker or earphone.

In yet a further embodiment, an array of crystals enables imaging of the operational site from the viewpoint of the fingertip.

In still yet another embodiment, ultrasound energy further allows the modality to be used in treatment of lesions. Solid organs, like the kidney and liver as well as soft tissue like the breast or for that matter, any place where lesions or cellular necrosis identification is desired, are within the teachings of this document.

It is also understood that the Doppler, ultrasound imaging and ultrasound therapy could be presented in individual or in any modality combinations. The output from the device could also be presented in numerous forms. The imaging and therapeutic applications may be presented on a screen of an ultrasound machine or independent monitor. Typically the monitor would be on the ultrasound machine or room monitor but could work with a smaller screen worn by the user if desired. Ideally the images could be integrated into a transmitter that would remove the cord tethering the finger device.

### BRIEF DESCRIPTION OF THE FIGURES

These and other features, aspects, and advantages of the invention will become more readily apparent with reference to the following detailed description of a presently preferred, but nonetheless illustrative, embodiment when read in conjunction with the accompanying drawings. The drawings referred to herein will be understood as not being drawn to scale, except if specifically noted, the emphasis instead being placed upon illustrating the principles of the invention. In the accompanying drawings:

FIG. 1 is a partially sectioned perspective view of a HALS operation using a Doppler ultrasound sensor to monitor blood flow in accordance with the present invention;

FIGS. 2a-c are perspective views of alternate embodiments of a Doppler sensor positioned at the tip of a surgeon's finger;

FIG. 3 is a sectioned view of the finger device with an ultrasonic transducer and ultrasonic receiver;

FIG. 4 is a perspective view of a finger-mounted ultrasonic sensor electrically connected to a circuit box and strapping means for attaching the circuit box to the surgeon;

FIGS. 5a-c are perspective views of alternate embodiments of an ultrasound imaging sensor with one or more crystals to form an array; and

FIG. 6 is a sectioned view of the finger device shown in FIG. 5.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

Further, it is understood that any one or more of the following-described embodiments, expressions of embodiments, examples, methods, etc. can be combined with any one or more of the other following-described embodiments, expressions of embodiments, examples, methods, etc.

While the methods and apparatus of the present invention are generally applicable to the performance of these surgical procedures during any operation, they are particularly applicable to their performance during Hand Assisted Laparoscopic Surgery (HALS) and, accordingly, will be described herein with reference to this invention.

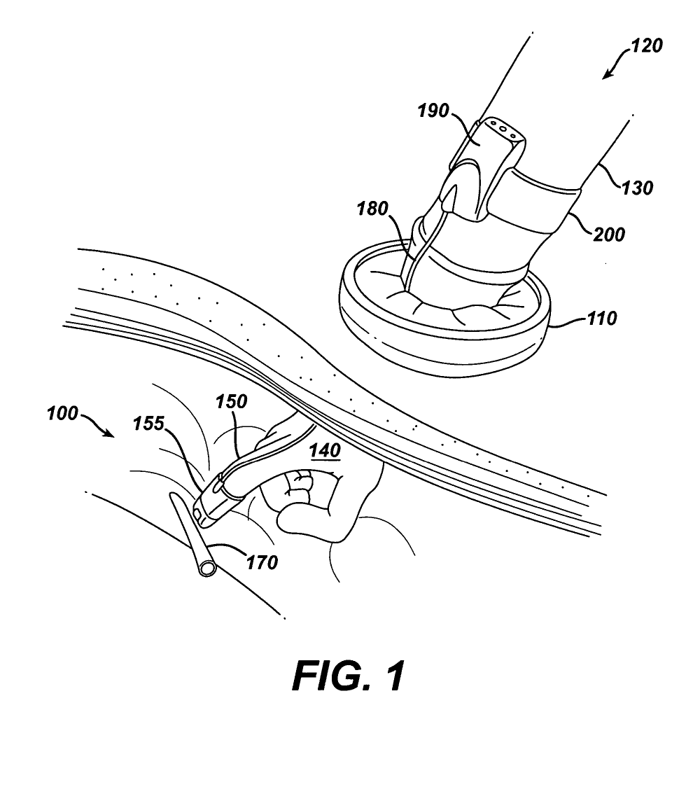

Referring now to FIG. 1, the environment for performing an endoscopic surgical procedure within an abdomen 100 is illustrated. A means for providing hand access, such as a lap disc 110, for example, model LD111 available from Ethicon Endo-Surgery, Cincinnati, Ohio, is placed into the abdominal wall. A surgeon 120 places his arm 130 and gloved hand 140 through the lap disc 110 and into the abdomen 100. The index finger 150 (any finger can be used) is capped with a finger device with an ultrasonic sensor 155. The finger device with ultrasonic sensor 155 is pressed against an operative field 170. Wires 180 connect to the circuitry box 190 mounted to the surgeon's arm 130 by a strapping means 200, such as Velcro, elastic, buckle or any conventional fastening means apparent to those skilled in the art.

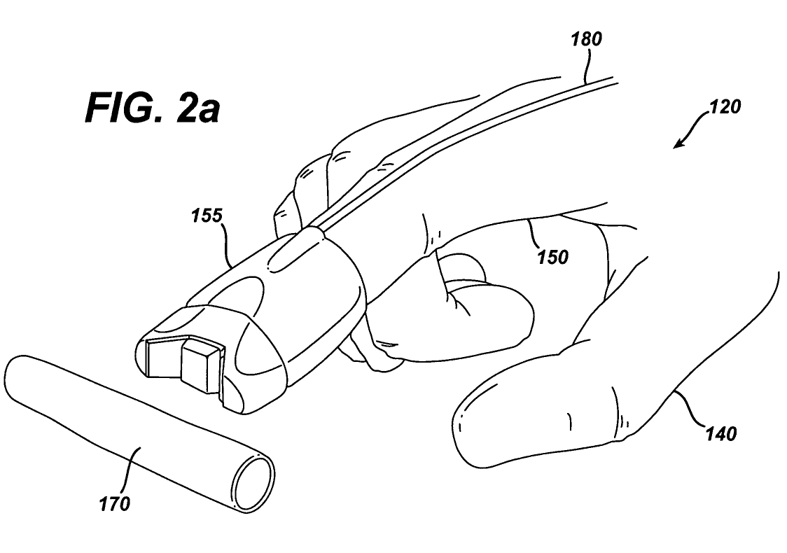

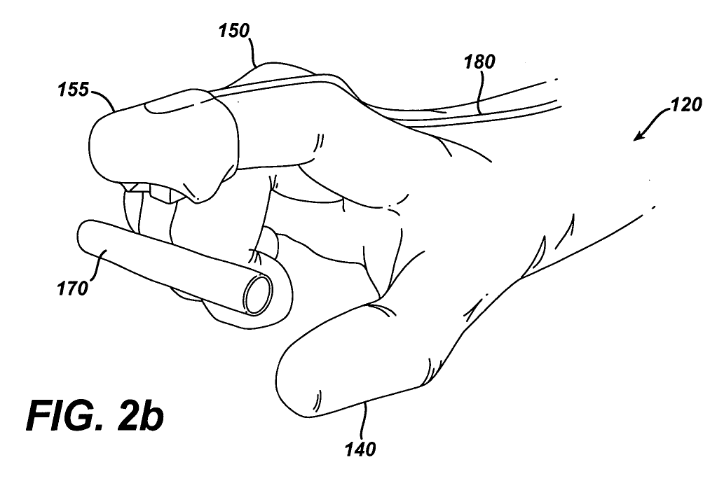

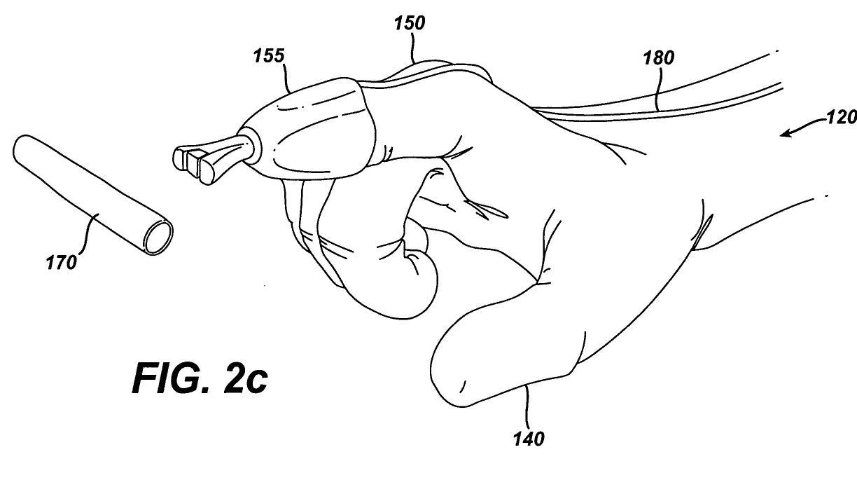

In FIG. 2a Doppler-sensor device approaches a vessel in an operative field 170 to sense its flow characteristics. FIGS. 2b and 2c illustrate alternate embodiments of incorporating the ultrasonic transducer of sensor 160 to the side, or finger pad of the fingertip, or as an extension of the fingertip.

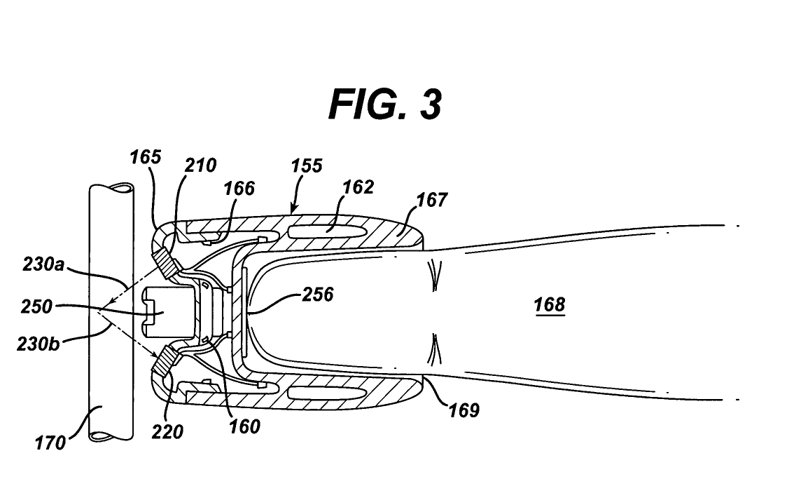

In FIG. 3, an ultrasonic sensor 155 comprises two subcomponents, the ultrasonic transducer 160 and the finger interface element 167. The ultrasonic transducer 160 comprises an ultrasonic transmitter 210 and an ultrasonic receiver 220 for directing and receiving ultrasonic energy to and from the operative field 170. The distal most surface of the fingertip sensor 155 supports the ultrasonic transmitter 210 and the ultrasonic receiver 220. The path of the ultrasonic energy for this embodiment of the invention is represented by the arrowed paths 230a and 230b. Acoustic lenses and matching layers may also be utilized with a transmitter and/or a receiver to direct ultrasonic energy to and from the operative field 170. The acoustic lenses may be made from a number of materials well known in the art to focus the ultrasonic energy as described and shown.

The fingertip ultrasonic sensor 155 further comprises a finger interface element 167 having an opening 169 for releasably receiving a surgeon's fingertip 168. Preferably, opening 169 is constructed to compressively engage the surgeon's fingertip 168. Opening 169 may also have a friction material on its internal surface to provide further gripping capabilities to secure the surgeon's fingertip 168 within opening 169. Preferably, finger interface element 167 comprises a mounting means, such as a channel 162 for receiving a securing element, such as a strap, to securely fasten the finger interface element 167 to the surgeon's finger 168.

For ease of manufacturing, the finger interface element 167 releasably connects with a mounting bracket 165 for mounting the ultrasonic transducer 210 and receiver 220 through conventional snap catches 166, detents or press fit means. Alternatively, interface element 167 and bracket 165 may be molded as one piece.

Also shown in FIG. 3 is a pressure switch 250 and tactile transducer 256. Pressure switch 250 enables completion of the circuit, discussed below. Tactile transducer 256 is located at the distal portion of opening 169 to allow the surgeon to gain an increased sensitivity to the pulsing of any contacted vessels, such as vessel 170. The tactile transducer 256 may be operated at a frequency of approximately 5 kilohertz.

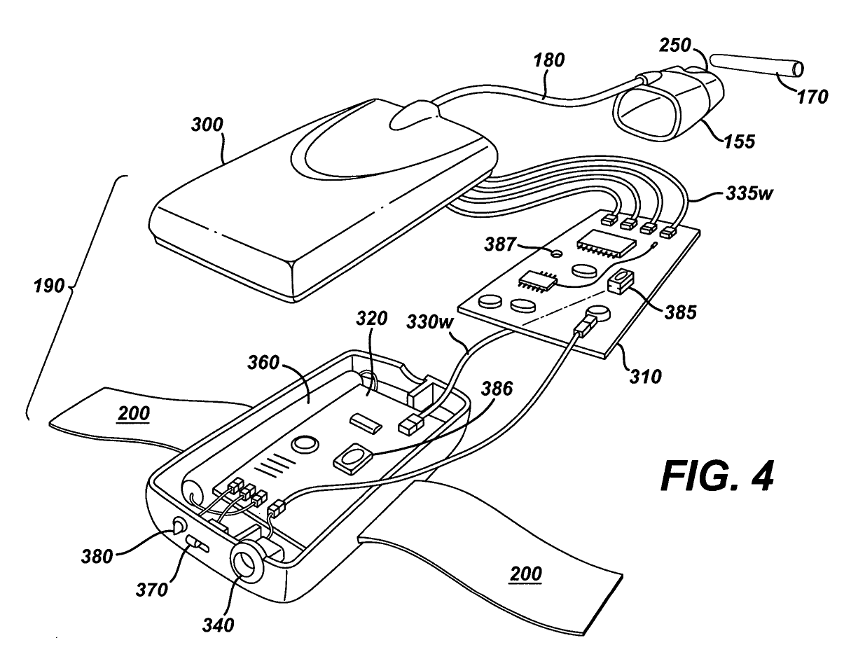

FIG. 4 is a perspective view of the circuit box 190 and strapping element showing the cover 300 offset to reveal structural details of the ultrasonic transducers incorporated therein. Whatever the form of the ultrasonic transducer, an appropriate circuit is provided for activating the transducer to transmit ultrasonic energy to the operational field as directed by the fingertip ultrasound sensor 155. The circuit also provides for receiving signals generated by the receiver 220 in response to received ultrasonic energy that is reflected from the operational field and for analyzing those signals. Since the circuit is a conventional circuit design as far as transmission and reception of ultrasonic energy and processing of the resulting signals is concerned, it will be described herein only with reference to its assembly and packaging which permits it to be readily combined with the ultrasonic sensor 155.

A representative circuit means for activating the transducer is a pressure switch 250 (FIG. 3) that is engaged when the operative field 170 is contacted. The circuitry is packaged on two printed circuit boards 310, 320. In general, the circuit boards 310, 320 are partitioned such that the upper printed circuit board 310 includes the circuitry for driving the ultrasonic transducer and the lower printed circuit board 320 includes the circuitry for receiving signals from the transducer. Accordingly, the upper printed circuit board 310 is connected to the lower circuit board 320 via wiring 330w and the lower printed circuit board 320 is connected to the ultrasonic receiver via wiring 335w.

In the illustrated embodiment, the circuit and transducer are constructed for operation at a frequency of approximately 20 megahertz. While it is apparent that other frequencies can be utilized in accordance with the present invention, the 20-megahertz frequency is used in the illustrated embodiments to better define the focus zone size and depth of penetration of the ultrasonic energy into the tissue. The circuitry on the boards 310, 320 is of a conventional design. Commercially available components may be surface and otherwise mounted to occupy a limited amount of board space on the boards 310, 320. The boards 310, 320 are also mounted in “piggy-back” fashion, with one board on top of the other to compact the circuitry further and conserve space within the circuitry box 190. While external circuitry can be utilized in the present invention, the compact arrangement illustrated is preferred since it forms a compact, self-contained enclosure.

In the illustrated embodiment, the circuitry on the boards 310, 320 is operated by power from a battery 360 mounted parallel and adjacent to the boards 310, 320. The battery 360 can be rechargeable in the event the ultrasonic sensor 155 is manufactured to be reusable. For a rechargeable battery, recharging can take place through the jack 340. Alternately, power for the circuit can be provided directly through the jack 340 with elimination of the battery 360.

More likely is the provision of a disposable device; the battery 360 is selected for power levels available from the battery and its shelf life. Currently, for disposable instruments, alkaline, lithium or silver oxide batteries provide sufficiently high power output and have long shelf life. To be sure that power is not drained from a battery of a battery-powered instrument, a power switch 370 is built into the circuitry box 190. To verify activation of the ultrasonic sensing system to the surgeon, a light emitting diode 380 or other indicator device located on the circuitry box 190 is activated while power is connected to the ultrasonic sensing system.

The circuitry on the printed circuit boards 310, 320 includes two potentiometers 385, 386 with the potentiometer 386 being accessed through an opening 387 in the board 310. One of the potentiometers 385, 386 is used to set the volume of an audible alerting device or the level of signal produced by the tactile transducer while the other one of the potentiometers 385, 386 is used to set a threshold level to which a Doppler signal is compared via comparator means included within the circuitry on the circuit boards 310, 320. If the Doppler signal exceeds the set threshold, then the user of the instrument is alerted either tactilely or audibly during that time. The using surgeon is able to detect venous flow, which generates a continuous alerting signal, and arterial flow, which generates a pulsating alerting signal. Further, a vessel such as the bile duct, which does not contain a fluid flowing at a sufficient velocity to generate a Doppler signal having amplitude in excess of the set threshold, may be determined. While it is contemplated that the potentiometers 385, 386 will be set and then sealed during production, it is possible to permit field adjustment by disassembly the circuitry box 190 or by providing openings (not shown) through the circuitry box 190. Resilient plugs or the like can seal such openings, for example.

If the device is constructed and operable in accordance with the invention of the present application, a surgeon is able to concentrate on manipulating the device into proper positions. After such positioning, the surgeon can sense ultrasonically thereby extending and returning the surgeon's sense of feeling to determine the contents of the instruments' operational fields prior to performing the procedures.

Alternate alerting means of communicating the Doppler response may comprise a set of headphones, a speaker or the like (not shown) which can be coupled to the circuitry on the boards 310, 320 by means of an electrical jack, which is mounted in the base of the circuitry box 190. It is also possible to incorporate a sound source directly into the circuitry box 190, which would further simplify the structure of the instrument when audible alerting is used.

Also shown in FIG. 4 is a strapping means 200 that enables the circuit box 190 to be conveniently placed on the surgeons arm. The specific closure means can be accomplished in numerous well-known ways for example Velcro or a buckle. Also well known are alternative mounting means such as belt or pocket clips. If desired, the circuit box 190 could be placed in some location other than on the surgeon.

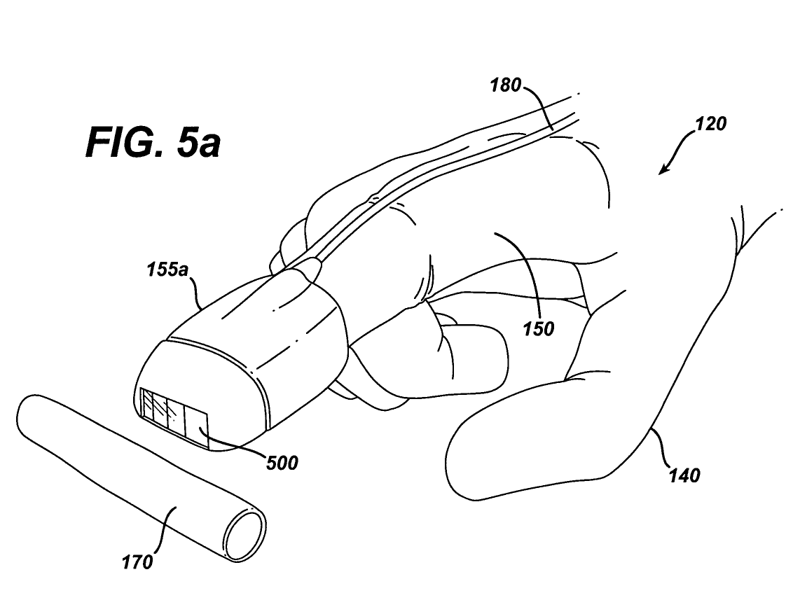

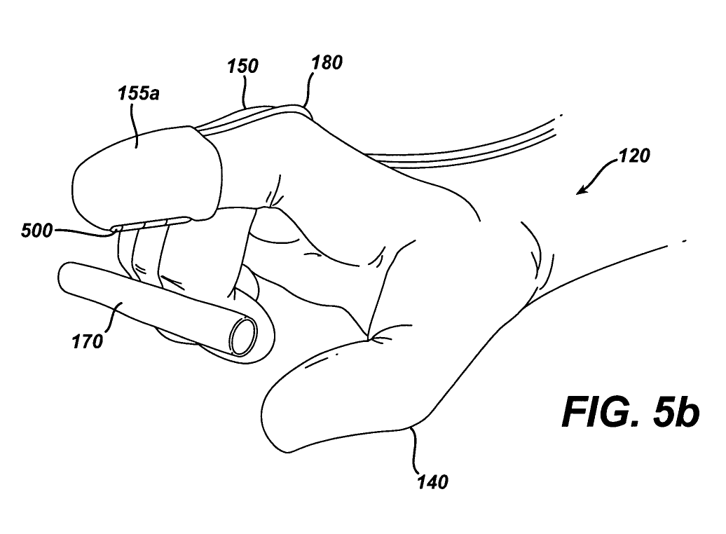

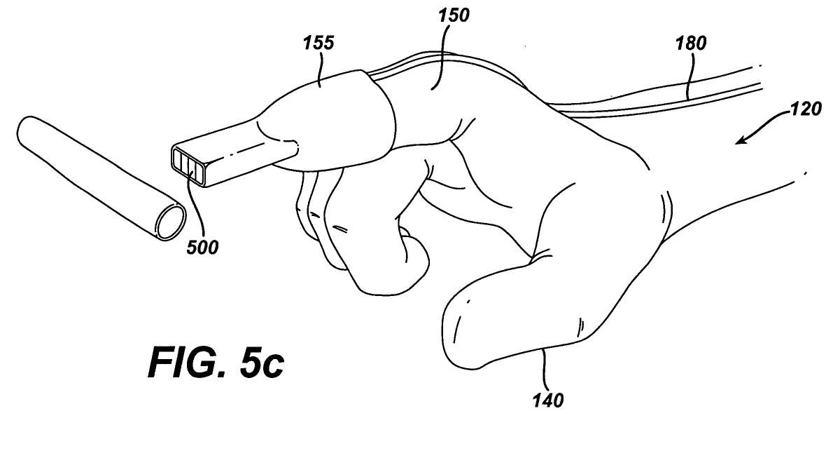

FIG. 5a is a perspective view of a device showing an ultrasound imaging sensor 155a with one or more crystals to form an ultrasound transducer array 500 on the distal end of a finger 150. The imaging sensor device 155a approaches an operative field 170 to image the tissue's characteristics. A representative ultrasonic transducer array is described in U.S. Pat. No. 6,050,943, and assigned to Guided Therapy Systems, Inc., the contents of which are hereby incorporated herein by reference.

FIG. 5b-c are alternate configurations to incorporate the ultrasonic transducer array into the side or finger pad or a distal extension of the imaging sensor device 155a.

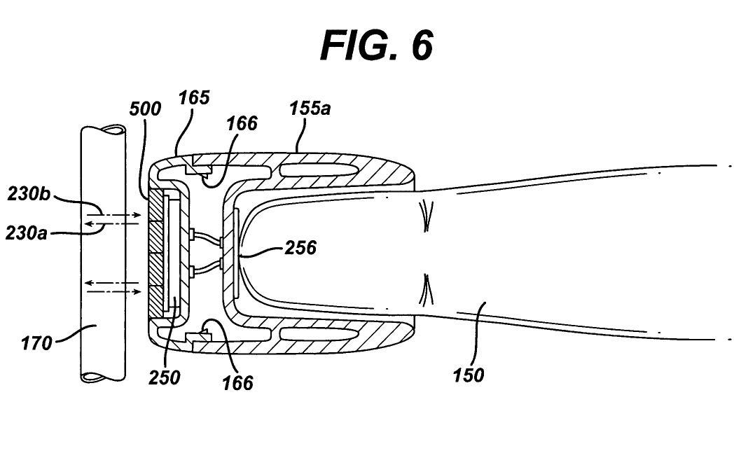

FIG. 6 is a perspective view of a fingertip ultrasound imaging sensor 155a where like reference numerals have the same description as corresponding numerals of FIG. 3. Ultrasonic transducer array 500 performs both transmitter and receiver functions. The path of the ultrasonic energy for this embodiment of the invention is represented by the arrowed paths 230a and 230b. Acoustic lenses and/or matching layers may also be utilized with a transmitter/receiver array to direct ultrasonic energy to and from the operative field 170 to improve imaging quality or therapeutic effect (discussed below). The acoustic lenses can be made from a number of materials well known in the art to focus the ultrasonic energy as described and shown. Accordingly, the acoustic lenses 220 and 230 will not be further described herein.

Also shown in FIG. 6 is a pressure switch 250. The pressure switch 250 enables completion of the circuit and image transducer upon contact of the ultrasound imaging sensor 155a with the operative field 170.

FIGS. 5 and 6 also represent an ultrasound imaging and/or therapy device that would enable the ultrasound energy to be focus to enable a therapeutic effect on the operative field 170. The therapeutic effect could be the treatment of lesions or solid organs like the kidney and liver as well as soft tissue like the breast or for that matter, any place where lesions or cellular necrosis is desired is within the teachings of this document. The surgeon may first image the tissue by moving the finger or incorporating a mechanism that would move the array while the finger was held in position. After an image is obtained the surgeon may then adjust the power setting of the transducer array 500 to ablate the identified tissue.

While the methods for performing ultrasonically assisted surgical procedures in accordance with the invention of the present application should be apparent from the foregoing description of illustrative embodiments of the invention, an illustrative method of such performance will now be described for sake of clarity. The method is for operating a device having a distal end for sensing an operational field and a means for activating performance within the operational field. Ultrasonic energy is transmitted to the operational field of the surgical instrument and reflected from the contents of the operational field. The ultrasonic energy reflected from the operational field of the device is received and Doppler signals representative of the contents of the operational field are generated in response to the received ultrasonic energy. The Doppler signals are analyzed to determine the nature of the contents of the operational field of the surgical instrument and the user of the surgical instrument is informed of the contents of the operational field. If the contents of the operational field are confirmed as being appropriate, the surgeon is confident to proceed with the procedure at hand.

While preferred embodiments of the present invention have been shown and described herein, it will be obvious to those skilled in the art that such embodiments are provided by way of example only. In addition, it should be understood that every structure described above has a function and such structure can be referred to as a means for performing that function. Numerous variations, changes, and substitutions will now occur to those skilled in the art without departing from the invention. Accordingly, it is intended that the invention be limited only by the spirit and scope of the appended claims.

What is claimed is:

1. A fingertip-mounted minimally invasive surgical instrument comprising:
a) a finger mount, having a proximal and distal end, and a cavity for releasably receiving a fingertip; and
b) an ultrasonic transducer and receiver positioned on the distal end of the finger mount.

2. The fingertip-mounted minimally invasive surgical instrument of claim 1, further comprising a pressure switch located at the distal end of the finger mount.

3. The fingertip-mounted minimally invasive surgical instrument of claim 1, further comprising a feedback transducer positioned within the cavity.

4. The surgical instrument of claim 1, wherein the transducer is a crystal array.

5. A method of performing a minimally invasive surgical procedure in a patient comprising:
a) creating an incision to permit hand access within the patient;
b) introducing a hand instrument comprising:
i) a finger mount, having a proximal and distal end, and a cavity for releasably receiving a fingertip; and
ii) an ultrasonic transducer and receiver positioned on the distal end of the finger mount; and
c) actuating the transducer to sense a operational site within the patient.

6. The method of claim 5 further comprising the step of releasably engaging a finger with the hand instrument.

7. The method of claim 5 further comprising the step of activating the transducer to image the surgical site

8. The method of claim 5 further comprising the step of actuating the transducer to provide therapeutic effects to the surgical site.
